(19) United States
(12) Patent Application Publication
(10) Pub. No.: US 2003/0181510 A1
(43) Pub. Date: Sep. 25, 2003

(54) Inhibition of muscle regeneration following myectomy

(76) Inventors: Robert Baker, Lexington, KY (US); Philip Bonner, Lexington, KY (US); Shardan Radmanesh, Lexington, KY (US)

Correspondence Address: Stockwell & Associates, PSC, Suite 201, 861 Corporate Drive, Lexington, KY, 40503, US

(21) Appl. No.: 10/392,649

(22) Filed: Mar. 19, 2003

Related U.S. Application Data
(60) Provisional application No. 60/365,886, filed on Mar. 19, 2002.

(51) Int. Cl. A61K 31/382; A61K 31/353

(52) U.S. Cl. 514/432; 514/456

(57) ABSTRACT

A method of inhibiting skeletal muscle regeneration following myectomy is disclosed. The method involves administering a tyrosine kinase inhibitor to an animal following myectomy of at least one muscle. Genistein is a preferred tyrosine kinase inhibitor under the present invention.

### CROSS-REFERENCE TO RELATED APPLICATIONS

This application claims priority from provisional application Ser. No. 60/365,886 filed Mar. 19, 2002.

### STATEMENT REGARDING FEDERALLY SPONSORED RESEARCH

N/A

### TECHNICAL FIELD OF THE INVENTION

The present invention relates to a method for the inhibition of skeletal muscle regeneration following myectomy.

### BRIEF SUMMARY OF THE INVENTION

The present invention relates to a method for the therapeutic inhibition of skeletal muscle regeneration following intentional myectomy by a medical professional, including myectomy for the treatment of skeletal muscle disorders, neurological disorders affecting skeletal muscles, and myectomy for cosmetic purposes.

The method involves the administration of an inhibitor of the protein tyrosine kinase pathway. Preferably, the inhibitor of the protein tyrosine kinase pathway is a compound of formula:

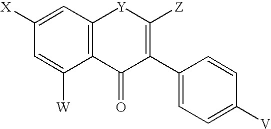

wherein V, W and X are selected from the group consisting of hydro, hydroxyl, alkoxy, halo, an ester, an ether, a carboxylic acid group, a pharmaceutically acceptable salt of a carboxylic acid group, and —SR, in which R is hydrogen or an alkyl group, and Y is selected from the group consisting of oxygen, sulfur, C(OH), and C═O, and Z is selected from the group consisting of hydro and C(O)OR₁, wherein R₁ is an alkyl. Preferably, the alkoxy is a C₁-C₆ alkoxy. Preferably, the halo is fluorine, chlorine or bromine. Preferably, the ester is a C₁-C₆ ester. Preferably, the ether is a C₁-C₆ ether. Preferred pharmaceutically acceptable salts of the carboxylic acid group include sodium and potassium salts. Preferably, the alkyl groups are C₁-C₆ alkyl groups. Desirably, the protein tyrosine kinase pathway inhibitor is genistein.

### DETAILED DESCRIPTION OF THE INVENTION

The present invention is predicated on the discovery that that an inhibitor of the protein tyrosine kinase pathway, specifically genistein, is effective in preventing the division of skeletal muscle satellite cells and the subsequent formation of myotubes by skeletal muscle satellite cells. Accordingly, the present invention provides a method for the therapeutic inhibition of the regeneration of skeletal muscle following myectomy. By “inhibition” it is meant the prevention, in whole or in part, of the formation of new muscle tissue (“regeneration”) by the division and fusion of satellite cells.

By “therapeutic” it is meant the prevention of muscle regeneration following myectomy performed to achieve a medical purpose. By “myectomy” we mean the removal or damage of muscle tissue. Removal or damage may be accomplished through mechanical or laser excision, or through exposure to myotoxic chemicals such as bupivicaine or doxorubicin (adriamycin). These specific methods are mentioned by way of example, and are not meant to exclude other methods. One skilled in the art will recognize that other suitable methods of damaging muscle tissue exist, and that this invention may be practiced using all of these methods.

The use of myectomy is well known in the medical arts. For example, myectomy is an accepted treatment to alleviate the symptoms of the focal dystonias, which are well known neuromuscular disorders characterized by the abnormal contraction of skeletal muscle. Myectomy is also utilized in cosmetic surgery to reduce or remove wrinkles or folds in the skin caused by the muscles of the face.

It is also well known in the medical arts that in many circumstances myectomy stimulates a regenerative response in the muscle tissue. (Carlson B M, Faulkner J A, Med Sci Sports Exerc 1983;15(3):187-98) The regenerative response is mediated by the activity of muscle satellite cells. Damage to muscle tissue induces satellite cells to proliferate and fuse with other satellite cells. The fusion ultimately leads to the formation of myotubes which replace damaged muscle tissue. The replacement of damaged muscle tissue coincides with a restoration of muscle function. When myectomy has been performed for to treat a disorder or to achieve a cosmetic effect, the restoration of muscle function is not desired. Accordingly, it is desirable to devise a method of preventing the regenerative response of satellite cells following myectomy.

The method of the present invention comprises the administration of an inhibitor of the protein tyrosine kinase pathway in an amount sufficient to inhibit the regeneration of skeletal muscle. Any inhibitor of the protein tyrosine kinase pathway can be used in the method of the present invention as long as it is safe and efficacious. Herein, “PTK inhibitor” will be used to refer to such compounds and is intended to encompass all compounds that affect the protein tyrosine kinase pathway at any and all points in the pathway.

Preferably, the PTK inhibitor is genistein (5,7-dihydroxy-3-(4-hydroxyphenyl)-4H-1-benzopyran-4-one) or a pharmaceutically acceptable, protein tyrosine kinase pathway-inhibiting analogue or prodrug thereof or a pharmaceutically acceptable salt of any of the foregoing. Accordingly, the PTK inhibitor can be a compound of the following formula:

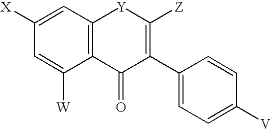

wherein V, W and X are selected from the group consisting of hyro, hydroxyl, alkoxy, halo, an ester, an ether, a carboxylic acid group, a pharmaceutically acceptable salt of a carboxylic acid group, and —SR, in which R is hydrogen or an alkyl group, and Y is selected from the group consisting of oxygen, sulfur, C(OH), and C═O, and Z is selected from the group consisting of hydro and C(O)OR₁, wherein R₁ is an alkyl. Preferably, the alkoxy is a C₁-C₆ alkoxy. Preferably, the halo is fluorine, chlorine or bromine. Preferably, the ester is a C₁-C₆ ester. Preferably, the ether is a C₁-C₆ ether. Preferred pharmaceutically acceptable salts of the carboxylic acid group include sodium and potassium salts. Preferably, the alkyl groups are C₁-C₆ alkyl groups. Desirably, the protein tyrosine kinase pathway inhibitor is genistein.

The prodrug can be any pharmaceutically acceptable prodrug of genistein, a protein tyrosine kinase pathway-inhibiting analogue of genistein, or a pharmaceutically acceptable salt of either of the foregoing. One of ordinary skill in the art will appreciate, however, that the prodrug used must be one that can be converted to an active PTK inhibitor. A preferred prodrug is a prodrug that increases the lipid solubility of genistein, a protein tyrosine kinase pathway-inhibiting analogue of genistein, or a pharmaceutically acceptable salt of either of the foregoing. A preferred prodrug is one in which one or more of V, W and X are independently derivatized with an ester, such as pivalic acid.

Compounds of the above formula are widely available commercially. For example, genistein is available from LC Laboratories (Woburn, Mass.). Those compounds that are not commercially available can be readily prepared using organic synthesis methods known in the art.

Whether or not a particular analogue, prodrug or pharmaceutically acceptable salt of a compound in accordance with the present invention can therapeutically inhibit the regeneration of skeletal muscle can be determined by its effect in the cell culture model used in Examples 1 and 2. Alternatively, analogues, prodrugs and pharmaceutically acceptable salts of inhibitors of the protein tyrosine kinase pathway can be tested by in vivo studies of skeletal muscle regeneration using mammalian models of muscle regeneration known in the art.

The PTK inhibitor can be bound to a suitable matrix, such as a polymeric matrix, if desired, for use in the present inventive method. Any of a wide range of polymers can be used in the context of the present invention provided that, if the polymer-bound compound is to be used in vivo, the polymer is biologically acceptable (see, e.g., U.S. Pat. Nos. 5,384,333 and 5,164,188).

An advantage of genistein is that it is very safe and efficacious. For example, genistein is a naturally occurring compound in some foods and populations of Southeast Asians are known to consume 70 mg/day of genistein without obvious ill effects (Soybean Utilization, Eds. Snyder & Kwon, p. 220).

The PTK inhibitor, which is preferably genistein, a protein tyrosine kinase pathway-inhibiting analogue of genistein, a protein tyrosine kinase pathway-inhibiting prodrug of genistein, or a pharmaceutically acceptable salt of any of the foregoing, can be administered in accordance with the present inventive method by any suitable route. Suitable routes of administration include systemic, such as orally, by injection or by the implantation of a delivery device such as the VITRASERT® implant manufactured by Bausch & Lomb.

The PTK inhibitor is preferably administered as soon as possible after myectomy has been performed. Treatment will depend, in part, upon the particular PTK inhibitor used, the amount of the PTK inhibitor administered, the route of administration, and the method of myectomy.

One skilled in the art will appreciate that suitable methods of administering a PTK inhibitor, which is useful in the present inventive method, are available. Although more than one route can be used to administer a particular PTK inhibitor, a particular route can provide a more immediate and more effective reaction than another route. Accordingly, the described routes of administration are merely exemplary and are in no way limiting.

The dose administered to an animal, particularly a human, in accordance with the present invention should be sufficient to effect the desired response in the animal over a reasonable time frame. One skilled in the art will recognize that dosage will depend upon a variety of factors, including the strength of the particular PTK inhibitor employed, the age, species, condition or disease state, and body weight of the animal. The size of the dose also will be determined by the route, timing and frequency of administration as well as the existence, nature, and extent of any adverse side effects that might accompany the administration of a particular PTK inhibitor and the desired physiological effect. It will be appreciated by one of ordinary skill in the art that various conditions or disease states, in particular, chronic conditions or disease states, may require prolonged treatment involving multiple administrations.

Suitable doses and dosage regimens can be determined by conventional range-finding techniques known to those of ordinary skill in the art. Generally, treatment is initiated with smaller dosages, which are less than the optimum dose of the compound. Thereafter, the dosage is increased by small increments until the optimum effect under the circumstances is reached. The present inventive method will typically involve the administration of from about 1 mg/kg/day to about 100 mg/kg/day, preferably from about 15 mg/kg/day to about 50 mg/kg/day, if administered systemically.

Compositions for use in the present inventive method preferably comprise a pharmaceutically acceptable carrier and an amount of a PTK inhibitor sufficient to inhibit myotube formation and satellite cell division in vivo. The carrier can be any of those conventionally used and is limited only by chemico-physical considerations, such as solubility and lack of reactivity with the compound, and by the route of administration. It will be appreciated by one of ordinary skill in the art that, in addition to the following described pharmaceutical compositions, the PTK inhibitor can be formulated as polymeric compositions, inclusion complexes, such as cyclodextrin inclusion complexes, liposomes, microspheres, microcapsules and the like (see, e.g., U.S. Pat. Nos. 4,997,652, 5,185,152 and 5,718,922).

The PTK inhibitor can be formulated as a pharmaceutically acceptable acid addition salt. Examples of pharmaceutically acceptable acid addition salts for use in the pharmaceutical composition include those derived from mineral acids, such as hydrochloric, hydrobromic, phosphoric, metaphosphoric, nitric and sulfuric acids, and organic acids, such as tartaric, acetic, citric, malic, lactic, fumaric, benzoic, glycolic, gluconic, succinic, and arylsulphonic, for example p-toluenesulphonic, acids.

The pharmaceutically acceptable excipients described herein, for example, vehicles, adjuvants, carriers or diluents, are well known to those who are skilled in the art and are readily available to the public. It is preferred that the pharmaceutically acceptable carrier be one which is chemically inert to the PTK inhibitor and one which has no detrimental side effects or toxicity under the conditions of use.

The choice of excipient will be determined in part by the particular PTK inhibitor, as well as by the particular method used to administer the composition. Accordingly, there are a wide variety of suitable formulations of the pharmaceutical composition of the present invention. The following formulations are merely exemplary and are in no way limiting.

Injectable formulations are among those that are preferred in accordance with the present inventive method. The requirements for effective pharmaceutically carriers for injectable compositions are well known to those of ordinary skill in the art (see Pharmaceutics and Pharmacy Practice, J. B. Lippincott Co., Philadelphia, Pa., Banker and Chalmers, eds., pages 238-250 (1982), and ASHP Handbook on Injectable Drugs, Toissel, 4th ed., pages 622-630 (1986)). It is preferred that such injectable compositions be administered intramuscularly, intravenously, or intraperitoneally.

Topical formulations are well known to those of skill in the art. Such formulations are suitable in the context of the present invention for application to the skin. The use of patches, ophthalmic solutions (see, e.g., U.S. Pat. No. 5,710,182) and ointments, e.g., eye drops, is also within the skill in the art.

Formulations suitable for oral administration can consist of (a) liquid solutions, such as an effective amount of the compound dissolved in diluents, such as water, saline, or orange juice; (b) capsules, sachets, tablets, lozenges, and troches, each containing a predetermined amount of the active ingredient, as solids or granules; (c) powders; (d) suspensions in an appropriate liquid; and (e) suitable emulsions. Liquid formulations may include diluents, such as water and alcohols, for example, ethanol, benzyl alcohol, and the polyethylene alcohols, either with or without the addition of a pharmaceutically acceptable surfactant, suspending agent, or emulsifying agent. Capsule forms can be of the ordinary hard- or soft-shelled gelatin type containing, for example, surfactants, lubricants, and inert fillers, such as lactose, sucrose, calcium phosphate, and cornstarch. Tablet forms can include one or more of lactose, sucrose, mannitol, corn starch, potato starch, alginic acid, microcrystalline cellulose, acacia, gelatin, guar gum, colloidal silicon dioxide, croscarmellose sodium, talc, magnesium stearate, calcium stearate, zinc stearate, stearic acid, and other excipients, colorants, diluents, buffering agents, disintegrating agents, moistening agents, preservatives, flavoring agents, and pharmacologically compatible excipients. Lozenge forms can comprise the active ingredient in a flavor, usually sucrose and acacia or tragacanth, as well as pastilles comprising the active ingredient in an inert base, such as gelatin and glycerin, or sucrose and acacia, emulsions, gels, and the like containing, in addition to the active ingredient, such excipients as are known in the art.

Formulations suitable for parenteral administration include aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The inhibitor can be administered in a physiologically acceptable diluent in a pharmaceutical carrier, such as a sterile liquid or mixture of liquids, including water, saline, aqueous dextrose and related sugar solutions, an alcohol, such as ethanol, isopropanol, or hexadecyl alcohol, glycols, such as propylene glycol or polyethylene glycol, dimethylsulfoxide, glycerol ketals, such as 2,2-dimethyl-1,3-dioxolane-4-methanol, ethers, such as poly(ethyleneglycol) 400, an oil, a fatty acid, a fatty acid ester or glyceride, or an acetylated fatty acid glyceride, with or without the addition of a pharmaceutically acceptable surfactant, such as a soap or a detergent, suspending agent, such as pectin, carbomers, methylcellulose, hydroxypropylmethylcellulose, or carboxymethylcellulose, or emulsifying agents and other pharmaceutical adjuvants. Oils, which can be used in parenteral formulations include petroleum, animal, vegetable, or synthetic oils. Specific examples of oils include peanut, soybean, sesame, cottonseed, corn, olive, petrolatum, and mineral.

Suitable fatty acids for use in parenteral formulations include oleic acid, stearic acid, and isostearic acid. Ethyl oleate and isopropyl myristate are examples of suitable fatty acid esters.

Suitable soaps for use in parenteral formulations include fatty alkali metals, ammonium, and triethanolamine salts, and suitable detergents include (a) cationic detergents such as, for example, dimethyl dialkyl ammonium halides, and alkyl pyridinium halides, (b) anionic detergents such as, for example, alkyl, aryl, and olefin sulfonates, alkyl, olefin, ether, and monoglyceride sulfates, and sulfosuccinates, (c) nonionic detergents such as, for example, fatty amine oxides, fatty acid alkanolamides, and polyoxyethylenepolypropylene copolymers, (d) amphoteric detergents such as, for example, alkyl-p-aminopropionates, and 2-alkyl-imidazoline quaternary ammonium salts, and (e) mixtures thereof.

The parenteral formulations will typically contain from about 0.5 to about 25% by weight of the active ingredient in solution. Preservatives and buffers may be used. In order to minimize or eliminate irritation at the site of injection, such compositions may contain one or more nonionic surfactants having a hydrophile-lipophile balance (HLB) of from about 12 to about 17.

The quantity of surfactant in such formulations will typically range from about 5 to about 15% by weight. Suitable surfactants include polyethylene sorbitan fatty acid esters, such as sorbitan monooleate and the high molecular weight adducts of ethylene oxide with a hydrophobic base, formed by the condensation of propylene oxide with propylene glycol. The parenteral formulations can be presented in unit-dose or multi-dose sealed containers, such as ampules and vials, and can be stored in a freeze-dried (lyophilized) condition requiring only the addition of the sterile liquid excipient, for example, water, for injections, immediately prior to use. Extemporaneous injection solutions and suspensions can be prepared from sterile powders, granules, and tablets of the kind previously described.

Such compositions can be formulated as sustained-release formulations or devices (see, e.g., U.S. Pat. No. 5,378,475). For example, gelatin, chondroitin sulfate, a polyphosphoester, such as bis-2-hydroxyethyl-terephthalate (BHET), or a polylactic-glycolic acid (in various proportions) can be used to formulate sustained-release formulations. Implants (see, e.g., U.S. Pat. Nos. 5,443,505, 4,853,224 and 4,997,652), devices (see, e.g., U.S. Pat. Nos. 5,554,187, 4,863,457, 5,098,443 and 5,725,493), such as an implantable device, e.g., a mechanical reservoir, an intraocular device or an extraocular device with an intraocular conduit (e.g., 100. mu. −1 mm in diameter), or an implant or a device comprised of a polymeric composition as described above, can be used.

The present inventive method also can involve the co-administration of other pharmaceutically active compounds. By “co-administration” is meant administration before, concurrently with, e.g., in combination with the PTK inhibitor in the same formulation or in separate formulations, or after administration of a PTK inhibitor as described above. For example, corticosteroids, e.g., prednisone, methylprednisolone, dexamethasone, or triamcinalone acetinide, or noncorticosteroid anti-inflammatory compounds, such as ibuprofen or flubiproben, can be co-administered. Similarly, vitamins and minerals, e.g., zinc, anti-oxidants, e.g., carotenoids (such as a xanthophyll carotenoid like zeaxanthin or lutein), and micronutrients can be co-administered. In addition, other types of inhibitors of the protein tyrosine kinase pathway, which include natural protein tyrosine kinase inhibitors like quercetin, lavendustin A, erbstatin and herbimycin A, and synthetic protein tyrosine kinase inhibitors like tyrphostins (e.g., AG490, AG17, AG213 (RG50864), AG18, AG82, AG494, AG825, AG879, AG1112, AG1296, AG1478, AG126, RG13022, RG14620 and AG555), dihydroxy- and dimethoxybenzylidene malononitrile, analogs of lavendustin A (e.g., AG814 and AG957), quinazolines (e.g., AG1478), 4,5-dianilinophalimides, and thiazolidinediones, can be co-administered with genistein or an analogue, prodrug or pharmaceutically acceptable salt thereof (see Levitzki et al., Science 267: 1782-1788 (1995), and Cunningham et al., Anti-Cancer Drug Design 7: 365-384 (1992)). In this regard, potentially useful derivatives of genistein include those set forth in Mazurek et al., U.S. Pat. No. 5,637,703. Neutralizing proteins to growth factors, such as a monoclonal antibody that is specific for a given growth factor, e.g., VEGF (for an example, see Aiello et al., PNAS USA 92: 10457-10461 (1995)), or phosphotyrosine (Dhar et al., Mol. Pharmacol 37: 519-525 (1990)), can be co-administered. Other various compounds that can be co-administered include inhibitors of protein kinase C (see, e.g., U.S. Pat. Nos. 5,719,175 and 5,710,145), cytokine modulators, an endothelial cell-specific inhibitor of proliferation, e.g., thrombospondins, an endothelial cell-specific inhibitory growth factor, e.g., TNF.alpha., an anti-proliferative peptide, e.g., SPARC and prolferin-like peptides, a glutamate receptor antagonist, aminoguanidine, an angiotensin-converting enzyme inhibitor, e.g., angiotensin II, calcium channel blockers, .psi.-tectorigenin, ST638, somatostatin analogues, e.g., SMS 201-995, monosialoganglioside GM1, ticlopidine, neurotrophic growth factors, methyl-2,5-dihydroxycinnamate, an angiogenesis inhibitor, e.g., recombinant EPO, a sulphonylurea oral hypoglycemic agent, e.g., gliclazide (non-insulin-dependent diabetes), ST638 (Asahi et al., FEBS Letter 309: 10-14 (1992)), thalidomide, nicardipine hydrochloride, aspirin, piceatannol, staurosporine, adriamycin, epiderstatin, (+)-aeroplysinin-1, phenazocine, halomethyl ketones, anti-lipidemic agents, e.g., etofibrate, chlorpromazine and spinghosines, aldose reductase inhibitors, such as tolrestat, SPR-210, sorbinil or oxygen, and retinoic acid and analogues thereof (Burke et al., Drugs of the Future 17(2): 119-131 (1992); and Tomlinson et al., Pharmac. Ther. 54: 151-194 (1992)). Selenoindoles (2-thioindoles) and related disulfide selenides, such as those described in Dobrusin et al., U.S. Pat. No. 5,464,961, are useful protein tyrosine kinase inhibitors. Those patients that exhibit systemic fluid retention, such as that due to cardiovascular or renal disease and severe systemic hypertension, can be additionally treated with diuresis, dialysis, cardiac drugs and antihypertensive agents.

### EXAMPLES

The following examples further illustrate the present invention but, of course, should not be construed as in any way limiting its scope.

### Example 1

This example demonstrates that genistein inhibits fusion of satellite cells.

Method: Thigh muscle tissue was removed from post-natal day 20-30 mouse pups and the tissue reduced to single cells by a method based largely on a protocol by DiMario and Strohman (DiMario, J. and Strohman, R. C.; 1988). Gastrocnemius and soleus muscles were dissected free of surrounding tissue and finely minced with scissors. The minced tissue was mixed with 0.10% crude collagenase and incubated at 37° C. for 30 minutes with occasional trituration through the bore of a 5 ml glass pipette to break up tissue pieces. The collagenase-tissue slurry was centrifuged briefly to pellet the tissue pieces, muscle fibers, and cells. The pellet was resuspended in 0.175% trypsin in a calcium-magnesium-free physiological saline solution and incubated at 37° C. for 20-30 minutes to release satellite cells from skeletal muscle fiber basal laminae. The trypsinized tissue suspension was filtered through 20Πm-pore nylon mesh (Nitex) to remove tissue pieces and cell clumps and the filtrate was spun at 2-3,000 ×g for 3 minutes to pellet the cells. The cell-containing pellet was resuspended in culture medium to block further protease action and the cells added to 35 mm collagen-coated tissue culture dishes and incubated at 38° C. and 100% humidity. After 3 to 5 days of primary culture the cells were removed from the culture dishes following incubation with 0.05% trypsin. The number of cells was determined and aliquots were appropriately diluted with culture medium so that a known number of satellite cells (80,000) could be added to additional 35 mm collagen-coated dishes for analysis of cell proliferation and fusion.

The culture medium used to support growth and differentiation of mouse primary satellite cells was FM. FM consists of 89% Ham's F-10 nutrient salts, 15% horse serum, 5% chick embryo extract, and 1% antibiotics (penicillin/streptomycin).

Three experimental groups were created by the addition of genistein to the culture medium on the first, second, or third day of culture. Genistein was dissolved in dimethlysulfoxide (DMSO) and included in the culture medium at a concentration of 100ΠM. A control group contained no genistein in the culture medium but did contain a concentration of DMSO identical to the experimental groups, 0.05%. Fresh culture medium with genistein/DMSO or DMSO alone was added to these cultures every day to enhance proliferation.

Fusion of satellite cells to form muscle fibers is generally accomplished by removing the source of growth factors in the culture medium. Two methods were employed: satellite cell cultures were not fed with fresh medium and the quickly-exhausted medium initiated fusion, similarly, cultures were fed with Δ-MEM medium devoid of embryo extract and containing reduced (5%) serum. As fusion of satellite cells to form myofibers began, the number of nuclei contained within mononucleated cells, primarily satellite cells, and those within myofibers were counted differentially at a magnification of 200×. The two culture media gave closely similar results.

A “fusion index” was prepared for each interval of each culture. The fusion index is defined as the mean percentage of nuclei found within myofibers in 10 random grids divided by the total number of nuclei in the same grids (satellite cell nuclei plus myofiber nuclei). This fraction was multiplied by 100 to give the percentage of nuclei within myofibers. The cell and fiber nuclei of random grids on each living culture dish were counted each day by phase contrast microscopy at 200×magnification.

Results: The results are summarized in FIG. 1. The addition of genistein inhibited the fusion of satellite cells, as demonstrated by the lower fusion index in genistein-treated cultures. The fusion index of control cultures was compared statistically to that of genistein-treated cultures at 3 and 5 days of fusion and found to be highly significantly different by Student's t-test. At day 3 of treatment significance of difference is P<0.002 and at day 5, P<0.0001.

### Example 2

This example demonstrates that genistein inhibits proliferation of satellite cells, the first step in muscle regeneration.

Method: To analyze satellite cell proliferation secondary satellite cell cultures derived from mouse gastrocnemius and soleus muscles prepared as in Example 1 were fed fresh medium every day. This generally accepted procedure prolongs proliferation and inhibits differentiation and fusion. Two experimental groups were formed by adding genistein to the culture medium, as in Example 1. The control group contained 0.05% DMSO in the culture medium, as did the experimental group, but did not contain genistein. Living cultures were placed on the stage of a phase-contrast microscope and the number of cells contained within an ocular grid was counted at 100× to 200× total magnification. This cell-counting procedure was performed daily through the sixth day of culture.

Results: Results are summarized in FIG. 2. Genistein eliminated the division of satellite cells when administered on either day1 or day 2. Satellite cells in the control group continued to divide. When examined statistically by Student's t-test the mean number of cells per reticle-grid of control cultures is highly significantly different from the means of genistein-treated cultures. When genistein was added on day 1 or on day 2 of culture the difference between experimental and control culture cell number per grid was significant at P<0.0001 one day after adding genistein and at P<0.00001 at days 2, 3, 4, and 5 after genistein addition.

While this invention has been described with an emphasis upon preferred embodiments, it will be obvious to those of ordinary skill in the art that variations of the preferred compounds and methods may be used and that it is intended that the invention may be practiced otherwise than as specifically described herein. Accordingly, this invention includes all modifications encompassed within the spirit and scope of the invention as defined by the following claims.

### REFERENCES

The following references, to the extent they provide exemplary procedural or other details supplementary to those set forth herein, are specifically incorporated herein by reference.

ASHP Handbook on Injectable Drugs, Toissel, 4th ed., pages 622-630 (1986)

Burke, Jr. “Protein-Tyrosine Kinase Inhibitors,” Drugs of the Future 17(2): 119-131 (1992).

Carlson B M, Faulkner J A, The regeneration of skeletal muscle fibers following injury: a review, Med. Sci. Sports Exerc. 15(3):187-98 (1983).

Cunningham et al., “Synthesis and Biological Evaluation of a Series of Flavones Designed as Inhibitors of Protein Tyrosine Kinase,” Anti-Cancer Drug Design 7: 365-384 (1992).

DiMario, J. & Strohman, R. C. (1988). Differentiation 39, 42-49.

Levitzki et al., Science 267: 1782-1788 (1995)

Pharmaceutics and Pharmacy Practice, J. B. Lippincott Co., Philadelphia, Pa., Banker and Chalmers, eds., pages 238-250 (1982), and).

Soybean Utilization, Eds. Snyder, H. E. and Kwon, T. W., Van Nostrand, New York, p.220.

Tomlinson et al., Pharmac. Ther. 54: 151-194 (1992)

What is claimed is:

1. A method of therapeutically treating an animal following myectomy of one or more muscles, which method comprises administering to said animal an inhibitor of the protein tyrosine kinase pathway, wherein said inhibitor is a compound of the form: 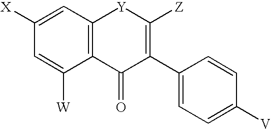
Wherein V, W and X are selected from the group of hydro, alkoxy, hydroxyl, halo, an ester, an ether, a carboxylic acid group, a pharmaceutically acceptable salt of a carboxylic acid group, and —SR, in which R is a hydrogen or an alkyl group, and Y is selected from the group consisting of oxygen, sulfur, C(OH), and C═O, and Z is selected from the group consisting of hydro and C(O)OR₁, wherein R₁ is an alkyl or a protein tyrosine kinase inhibiting prodrug, or a pharmaceutically acceptable salt thereof, in an amount sufficient to inhibit restoration of muscle function therapeutically.

2. The method of claim 1, wherein the halo group is selected from the group consisting of fluorine, chlorine and bromine.

3. The method of claim 1, wherein the ester is a C₁-C₆ ester.

4. The method of claim 1, wherein the ether is a C₁-C₆ ether.

5. The method of claim 1, wherein said pharmaceutically acceptable salt of a carboxylic acid group is a sodium salt or a potassium salt.

6. The method of claim 1, wherein the alkyl groups are C₁-C₆ alkyl groups and the alkoxy group is a C₁-C₆ alkoxy group.

7. The method of claim 1, wherein said inhibitor of the protein tyrosine kinase pathway is genistein.

8. The method of claim 7, wherein genistein is administered systemically.

9. The method of claim 8, wherein genistein is administered in an amount from about 1 mg/kg/day to about 100 mg/kg/day.

10. The method of claim 9, wherein genistein is administered in an amount from about 15 mg/kg/day to about 50 mg/kg/day.

11. The method of claim 8, wherein genistein is administered orally or by injection.

12. The method of claim 8, wherein said myectomy is performed to treat a dystonia.

13. The method of claim 8, wherein said myectomy is performed for cosmetic purposes.

14. The method of claim 8, wherein myectomy is performed through mechanical excision.

15. The method of claim 8, wherein myectomy is performed through laser excision.

16. The method of claim 8, wherein myectomy is performed through utilizing at least one myotoxic chemical.

17. The method of claim 16, wherein the myotoxic chemical is bupivicaine or doxorubicin.

18. The method of claim 8, wherein genistein is administered by the implantation of a delivery device containing genistein.

19. The method of claim 8, wherein genistein is administered by a topical formulation.
